(19) United States
(12) Patent Application Publication
(10) Pub. No.: US 2022/0370609 A1
(43) Pub. Date: Nov. 24, 2022

(54) Polymer complex for anticancer immune therapy based on ultrasound comprising oxalate derivatives and Method of preparation thereof

(71) Applicant: RESEARCH & BUSINESS FOUNDATION SUNGKYUNKWAN UNIVERSITY, Suwon-si (KR)

(72) Inventors: Jae Hyung PARK, Suwon-si (KR); Jueun JEON, Suwon-si (KR); Been YOON, Seoul (KR)

(73) Assignee: RESEARCH & BUSINESS FOUNDATION SUNGKYUNKWAN UNIVERSITY, Suwon-si (KR)

(21) Appl. No.: 17/748,197

(22) Filed: May 19, 2022

(30) Foreign Application Priority Data
May 20, 2021 (KR) 10-2021-0065088

(51) Int. Cl. A61K 41/00; A61K 31/765; A61K 47/10; A61K 47/22; A61K 47/36; A61P 35/00

(52) U.S. Cl. CPC A61K 41/0033; A61K 31/765; A61K 47/10; A61K 47/22; A61K 47/36; A61P 35/00

(57) ABSTRACT

The present invention relates to a polymer composite for ultrasound-based cancer immunotherapy, which comprises an peroxalate derivatives, and a preparation method thereof. The polymer composite according to the present invention is a structure in which the peroxalate derivatives are encapsulated in an amphipathic polymer compound in which a biocompatible polymer and a sonosensitizer are combined. The peroxalate derivatives produce free electrons and carbon dioxide (CO₂) by reaction with a high concentration of hydrogen peroxide (H₂O₂) in cancer tissue, the generated electrons raise the energy level of the sonosensitizer in the polymer composite to increase the amount of reactive oxygen species (ROS) production, thereby exhibiting an effect of increasing the death rate of cancer cells. In addition, by ultrasound treatment, immunogenic cell death (ICD) is induced due to the cavitation effect of the produced CO₂, so molecules capable of activating immune cells in cancer cells are released without damage to induce an immune response to cancer. Therefore, the polymer composite according to the present invention is expected to be effectively used as an ultrasound-based cancer immunotherapeutic agent.

### CROSS-REFERENCE TO RELATED APPLICATION

This application claims priority to and the benefit of Korean Patent Application No. 10-2021-0065088, filed on May 20, 2021, the disclosure of which is incorporated herein by reference in its entirety.

### BACKGROUND

### 1. Field of the Invention

The present invention relates to a polymer composite for ultrasound-based cancer immunotherapy, which comprises an peroxalate derivatives, and a preparation method thereof.

### 2. Discussion of Related Art

Cancer is one of the incurable diseases that mankind must solve, and huge capital is being invested in development to cure it worldwide, and cancer is the number one cause of death in Korea, with over 100,000 diagnosed cases and over 60,000 deaths annually.

Common anticancer therapies for cancer treatment include surgery, chemotherapy, and radiation therapy. Among these, patients who cannot easily undergo surgery or radiation therapy (approximately 50% of all cancer cases) and patients who have metastasized cancer are mainly treated with chemotherapy. However, due to drug resistance, recurrence, metastasis, and sequelae, it is important to develop cancer treatment technology that can minimize side effects.

Sonodynamic therapy (SDT) is a representative non-invasive cancer treatment method that uses ultrasound and a sonosensitizer to generate singlet oxygen (¹O₂) with cytotoxicity, thereby causing death and necrosis of cancer cells.

The strong hydrophobic nature of the sonosensitizer used for SDT has the disadvantage of not only an inefficient distribution behavior, but also low reactive oxygen species (ROS) generation efficiency at the in vivo. In addition, SDT-induced apoptosis is non-immunogenic cell death (ICD), and has a limitation in that a sufficient immune response cannot be induced because immunogenicity-associated molecules such as tumor associated antigens (TAAs) and damage-associated molecular patterns (DAMPs) are degraded in cells. In addition, SDT-induced cell necrosis is known to make fragments of tumor cells to partly induce antitumor immunity, but there is a limitation in that the single therapy may not elicit a sufficient acquired immune response.

Therefore, in order to overcome the limitations of SDT-based immunotherapy, studies are being conducted in which it is expected to improve the sonodynamic effect and the rupture of a cell membrane by inducing the cavitation effect of a gas using perfluorocarbon (PFC), but in the case of PFC, the stability of bubbles in the body is low, so side effects may occur due to indiscriminate vaporization in the body. Accordingly, there was a need for a study on how to maximize the SDT-based sonodynamic effect and an anticancer immune response without side effects.

Meanwhile, cancer immunotherapy is a method of treating cancer by increasing the immune responses against cancer cells or suppressing immune evasion, includes immune cell therapy, immune checkpoint inhibitors, therapeutic cancer vaccines, and therapeutic antibodies.

The present inventors developed a polymer composite that can overcome the limitations of low ROS production efficiency and no induction of a sufficient immune response in the conventional SDT as described above, and can be used as an ultrasound-based cancer immunotherapeutic agent by inducing ICD in response to a disease-specific environment, and the present invention was completed.

### SUMMARY OF THE INVENTION

The present inventors prepared a polymer composite in which peroxalate is encapsulated in an amphipathic polymer compound in which a hydrophilic biocompatible polymer and a hydrophobic sonosensitizer are combined, and confirmed that the polymer composite reinforces a sonodynamic effect and induces ICD.

Therefore, the present invention is directed to providing a polymer composite for ultrasound-based cancer immunotherapy, which comprises an peroxalate derivatives, characterized by the peroxalate derivatives being encapsulated in an amphipathic polymer compound in which a biocompatible polymer and a sonosensitizer are combined, and a preparation method thereof.

The present invention is also directed to providing a pharmaceutical composition for preventing or treating cancer, which comprises the polymer composite as an active ingredient.

However, technical problems to be solved in the present invention are not limited to the above-described problems, and other problems which are not described herein will be fully understood by those of ordinary skill in the art from the following descriptions.

To achieve the above purposes, the present invention provides a polymer composite for ultrasound-based cancer immunotherapy, which comprises peroxalate derivatives,

wherein the polymer composite is characterized by the peroxalate derivatives being encapsulated in an amphipathic polymer compound in which a biocompatible polymer and a porphyrin-based sonosensitizer are combined.

The present invention also provides a pharmaceutical composition for ultrasound-based cancer immunotherapy, which comprises the polymer composite as an active ingredient.

The present invention also provides a pharmaceutical composition for preventing or treating cancer, which comprises the polymer composite as an active ingredient.

The present invention also provides a method of preparing a polymer composite for ultrasound-based cancer immunotherapy, which comprises the following steps:

(a) mixing a porphyrin-based sonosensitizer solution and a biocompatible polymer solution to allow a reaction;

(b) adding the reaction solution to a dialysis membrane to perform dialysis;

(c) after dialysis, lyophilizing the amphipathic polymer compound solution in which the biocompatible polymer and the sonosensitizer are combined to prepare a powder; and

(d) encapsulating an peroxalate derivatives in the amphipathic polymer compound using an oil-in-water emulsion method.

In one embodiment of the present invention, the peroxalate derivative may be one or more selected from the group consisting of dibutyl oxalate, bis(2,4,6-trichlorophenyl) oxalate (TCPO), and bis[2,4,5-trichloro-6-(pentyloxycarbonyl)phenyl] oxalate (CPPO), but the present invention is not limited thereto.

In another embodiment of the present invention, the biocompatible polymer may be one or more selected from the group consisting of polyethylene glycol (PEG), carboxymethyl dextran (CMD), a dextran derivative, and hyaluronic acid, but the present invention is not limited thereto.

In still another embodiment of the present invention, the polyethylene glycol may have a molecular weight of 1 to 500 kDa, but the present invention is not limited thereto.

In yet another embodiment of the present invention, the porphyrin-based sonosensitizer may be one or more selected from the group consisting of verteporfin (VPF) and chlorin e6 (Ce6), but the present invention is not limited thereto.

In yet another embodiment of the present invention, in the polymer composite, the dry weight ratio of the sonosensitizer may be 0.05- to 0.5-fold that of the biocompatible polymer, but the present invention is not limited thereto.

In yet another embodiment of the present invention, in the polymer composite, the molar ratio of the sonosensitizer may be 0.2- to 5-fold that of the biocompatible polymer, but the present invention is not limited thereto.

In yet another embodiment of the present invention, the peroxalate derivatives may be encapsulated at a 1- to 1000-fold molar ratio, with respect to the amphipathic polymer compound in which the biocompatible polymer and the sonosensitizer are combined, but the present invention is not limited thereto.

In yet another embodiment of the present invention, the peroxalate derivatives may react with hydrogen peroxide (H₂O₂) in tumor tissue to increase reactive oxygen species (ROS) production of the sonosensitizer during ultrasound treatment, but the present invention is not limited thereto.

In yet another embodiment of the present invention, the peroxalate derivatives may react with H₂O₂ in tumor tissue to produce carbon dioxide (CO₂), but the present invention is not limited thereto.

In yet another embodiment of the present invention, the CO₂ may cause cavitation during ultrasound treatment, thereby inducing immunogenic cell death (ICD), but the present invention is not limited thereto.

In yet another embodiment of the present invention, the polymer composite may be released out of cells while at least one immunogenicity-associated protein selected from the group consisting of tumor-associated antigens (TAAs) and damage-associated molecular patterns (DAMPs) in cancer cells is not degraded, but the present invention is not limited thereto.

In yet another embodiment of the present invention, the pharmaceutical composition may further comprise an immune checkpoint inhibitor, but the present invention is not limited thereto.

In yet another embodiment of the present invention, the immune checkpoint inhibitor may be one or more selected from the group consisting of a PD-1 inhibitor, a PD-L1 inhibitor, and a CTLA-4 inhibitor, but the present invention is not limited thereto.

In yet another embodiment of the present invention, the cancer may be one or more selected from the group consisting of colorectal cancer, breast cancer, prostate cancer, melanoma, lung cancer, head and neck cancer, ovarian cancer, bladder cancer, stomach cancer, esophageal cancer, bile duct cancer, pancreatic cancer, liver cancer, cervical cancer, skin cancer, lymphoma, thyroid cancer, bone marrow cancer, endometrial cancer, and brain tumors, but the present invention is not limited thereto.

In yet another embodiment of the present invention, in (b) of the method of preparing a polymer composite for ultrasound-based cancer immunotherapy, the dialysis membrane may have a molecular weight cut off of 1 to 10 kDa, but the present invention is not limited thereto.

The present invention also provides an ultrasound-based cancer immunotherapy method, which comprises administering a composition comprising the polymer composite as an active ingredient to a subject.

The present invention also provides a use of a composition comprising the polymer composite as an active ingredient for ultrasound-based cancer immunotherapy.

The present invention also provides a use of a composition comprising the polymer composite as an active ingredient for preparing a drug for ultrasound-based cancer immunotherapy.

The present invention also provides a method of preventing or treating cancer, which comprises administering a composition comprising the polymer composite as an active ingredient to a subject.

The present invention also provides a use of a composition comprising the polymer composite as an active ingredient for preventing or treating cancer.

The present invention also provides a use of a composition comprising the polymer composite as an active ingredient for preparing a drug for preventing or treating cancer.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a schematic diagram showing the action of a polymer composite (Ox@PEG-VPF) comprising a peroxalate derivative according to one embodiment of the present invention;

FIG. 2 shows the structure of a conjugate of a polymer (PEG) and a sonosensitizer (VPF) according to one embodiment of the present invention and the ¹H-NMR result thereof;

FIG. 3A is a schematic diagram of the chemiluminescence resonance energy transfer phenomenon by the chemical reaction of an peroxalate derivatives according to one embodiment of the present invention and H₂O₂;

FIGS. 3B and 3C are views confirming chemiluminescence occurring depending on H₂O₂ treatment to a polymer composite comprising peroxalate derivatives according to one embodiment of the present invention;

FIG. 4 is a view confirming CO₂ generation in a colorectal cancer cell line (CT26) and a normal cell line (L929) by the treatment of a polymer composite comprising peroxalate derivatives according to one embodiment of the present invention;

FIGS. 5A to 5C are views confirming the CO₂ generation behavior of a polymer composite comprising peroxalate derivatives according to one embodiment of the present invention, wherein FIGS. 5A and 5C confirm CO₂ generation behavior depending on the presence of H₂O₂, and FIG. 5B confirms CO₂ generation behavior depending on peroxalate encapsulation;

FIGS. 6A and 6B are views confirming the CO₂ production of a polymer composite comprising a polymer-sonosensitizer conjugate and an peroxalate derivatives according to one embodiment of the present invention, confirmed using an ultrasound imaging device, in which FIG. 6A shows CO₂ production under a H₂O₂ mimicking condition of cancer tissue, and FIG. 6B shows CO₂ production in a colorectal cancer cell line CT26;

FIGS. 7A and 7B are graphs confirming cytotoxicity by the treatment of a polymer composite comprising a polymer-sonosensitizer conjugate and an peroxalate derivatives according to one embodiment of the present invention, wherein FIG. 7A is a graph confirming cytotoxicity in a colorectal cancer cell line CT26, and FIG. 7B is a graph confirming cytotoxicity in a normal cell line L929;

FIG. 8 is a graph confirming cytotoxicity in a colorectal cancer cell line CT26 according to one embodiment of the present invention by ultrasound irradiation alone;

FIG. 9 is a view confirming the CO₂ cavitation effect by H₂O₂ and ultrasound treatment in a polymer composite comprising peroxalate derivatives according to one embodiment of the present invention;

FIGS. 10A and 10B are views confirming the ROS generation ability of a polymer composite comprising peroxalate derivatives according to one embodiment of the present invention, in which FIG. 10A shows the result when ultrasound is not treated, and FIG. 10B shows the result when ultrasound is treated (in FIG. 10B, n=3, *p<0.05);

FIG. 11 is a view confirming the ability to generate ROS in cancer cells by ultrasound treatment of a polymer composite comprising peroxalate derivatives according to one embodiment of the present invention;

FIG. 12 is a view confirming the cancer cell death effect by the ultrasound treatment of a polymer composite comprising peroxalate derivatives according to one embodiment of the present invention (n=3, ****p<0.0001);

FIG. 13 is a view confirming the CO₂ cavitation effect exhibited by ultrasound treatment of a polymer composite comprising peroxalate derivatives according to one embodiment of the present invention and the occurrence of immunogenic cell death (ICD) caused thereby;

FIG. 14 is a view confirming whether ICD-associated proteins are released out of cells when a polymer composite comprising peroxalate derivatives according to one embodiment of the present invention is treated with ultrasound;

FIGS. 15A and 15B are views confirming the change in tumor size when tumor-bearing animal models are administered a polymer composite comprising peroxalate derivatives according to one embodiment of the present invention and then treated with ultrasound (in FIG. 15B, n=5, *p<0.05, **p<0.005);

FIG. 15C is a view confirming the survival rate of an animal when tumor-bearing animal models are administered a polymer composite comprising peroxalate derivatives according to one embodiment of the present invention and treated with ultrasound;

FIG. 15D is a view confirming the concentration of antitumor immunity-associated factors in blood after tumor-bearing animal models are administered a polymer composite comprising peroxalate derivatives according to one embodiment of the present invention and treated with ultrasound (n=5, ****p<0.0001);

FIGS. 16A and 16B are views confirming the change in tumor size when tumor-bearing animal models are administered a polymer composite comprising peroxalate derivatives according to one embodiment of the present invention, treated with ultrasound and co-administered an immune checkpoint inhibitor (in FIG. 16B, n=5, **p<0.005, ***p<0.0005);

FIG. 16C shows tumor weights after tumor-bearing animal models are administered a polymer composite comprising peroxalate derivatives according to one embodiment of the present invention, treated with ultrasound and co-administered an immune checkpoint inhibitor (n=5, **p<0.005, ****p<0.001); and

FIG. 16D is a view confirming the concentrations of antitumor immunity-associated factors present in tumor tissue after tumor-bearing animal models are administered a polymer composite comprising peroxalate derivative according to one embodiment of the present invention, treated with ultrasound and co-administered an immune checkpoint inhibitor (n=4, *p<0.05).

### DETAILED DESCRIPTION OF EXEMPLARY EMBODIMENTS

The present invention provides a polymer composite for ultrasound-based cancer immunotherapy, which comprises an peroxalate derivative, wherein the polymer composite is characterized by the peroxalate derivatives being encapsulated in an amphipathic polymer compound in which a biocompatible polymer and a porphyrin-based sonosensitizer are combined.

In the present invention, the peroxalate derivatives may be one or more selected from the group consisting of dibutyl oxalate, bis(2,4,6-trichlorophenyl) oxalate (TCPO), and bis[2,4,5-trichloro-6-(pentyloxycarbonyl)phenyl] oxalate (CPPO), and according to one example or experimental example of the present invention, the peroxalate derivative may be dibutyl oxalate, but the present invention is not limited thereto.

The “polymer” used herein refers to a molecule having a molecular weight of 1 kDa or more, and the “biocompatible polymer” refers to a polymer material that does not exhibit toxicity in the human body. In the present invention, the biocompatible polymer may be one or more selected from the group consisting of polyethylene glycol (PEG), carboxymethyl dextran (CMD), a dextran derivative, and hyaluronic acid. According to one example or experimental example of the present invention, the biocompatible polymer may be PEG, but the present invention is not limited thereto. Here, there is no limitation to the PEG as long as its molecular weight ranges from 1 to 500 kDa, but the present invention is not limited thereto. For example, the PEG may have a molecular weight of 1 to 400 kDa, 1 to 300 kDa, 1 to 200 kDa, 1 to 100 kDa, 1 to 50 kDa, 1 to 20 kDa, 1 to 10 kDa, 3 to 500 kDa, 3 to 300 kDa, 3 to 100 kDa, 3 to 50 kDa, 3 to 20 kDa, or 3 to 10 kDa.

The “ultrasound” used herein means a sound wave that exceeds a frequency of 16 Hz to 20 kHz, which is a frequency of sound waves that can be heard by the human ear in general, and high-intensity focused ultrasound introduces focused ultrasound that provides continuous and high-intensity ultrasound energy to the focus, and can exhibit an instantaneous thermal effect (65 to 100° C.), a cavitation effect, a mechanical effect, and a sonochemical effect depending on energy and frequency. While ultrasound is harmless when passing through human tissues, high-intensity focused ultrasound generates enough energy to cause coagulation necrosis and thermal cauterization regardless of a tissue type.

The “sonosensitizer” used herein means a material for increasing the sensitivity of cells to ultrasound in response to ultrasound or accelerating the treatment of a disease treatable by ultrasound, and the sonosensitizer may be porphyrin-based, and one or more selected from the group consisting of verteporfin (VPF) and chlorin e6 (Ce6), and according to one example or experimental example of the present invention, the sonosensitizer may be VPF, but the present invention is not limited thereto.

According to one example or experimental example of the present invention, a polymer composite for ultrasound-based cancer immunotherapy has a structure in which peroxalate derivatives is encapsulated in an amphipathic polymer compound in which a biocompatible polymer, PEG, and a sonosensitizer, VPF, are combined, and here, PEG and VPF, used as a biocompatible polymer and a sonosensitizer, respectively, are materials that have passed clinical trials, so they have the advantage of ensuring stability and simplifying a conjugation process due to the simple material structure.

The “ROS” used herein indicates a chemical species in which oxygen is chemically active, is very unstable and strongly oxidative. The ROS may be, for example, one or more selected from the group consisting of superoxide anion radicals, hydroxyl radicals (OH), hydrogen peroxide (H₂O₂), singlet oxygen (¹O₂), nitric oxide (NO), nitrogen dioxide (NO₂), ozone (O₃), and lipid peroxides, but the present invention is not limited thereto.

The “cavitation” used herein means a phenomenon in which, when a low-pressure region is created in a fluid, the gas contained in the fluid escapes from the fluid and is concentrated in a low-pressure region, resulting in an empty space created without fluid. Since the polymer composite has the encapsulated peroxalate derivatives, which are gas precursor, the peroxalate derivatives can react with H₂O₂ to form free electrons and CO₂, increase ROS production by increasing the energy level of the sonosensitizer due to the free electrons during ultrasound treatment, and induce immunogenic cell death by the rupture of the cell membrane by CO₂ collapse by cavitation.

The present invention also provides a pharmaceutical composition for ultrasound-based cancer immunotherapy, which comprises the polymer composite as an active ingredient.

In the present invention, the pharmaceutical composition may further comprise an immune checkpoint inhibitor, but the present invention is not limited thereto.

In the present invention, the “immune checkpoint inhibitor” is a material that attacks cancer cells by activating T cells by blocking the activity of an immune checkpoint protein involved in T cell inhibition, and may be, for example, one or more selected from the group consisting of a PD-1 inhibitor including atezolizumab, a PD-L1 inhibitor including pembrolizumab, and a CTLA-4 inhibitor including ipilimumab. According to one example or experimental example of the present invention, the immune checkpoint inhibitor may be an anti PD-1 antibody, but the present invention is not limited thereto.

The present invention also provides a pharmaceutical composition for preventing or treating cancer, which comprises the polymer composite as an active ingredient.

The present invention also provides an ultrasound-based cancer immunotherapy method, which comprises administering a composition comprising the polymer composite as an active ingredient to a subject.

The present invention also provides a use of a composition comprising the polymer composite as an active ingredient for ultrasound-based cancer immunotherapy.

The present invention also provides a use of a composition comprising the polymer composite as an active ingredient for preparing a drug for ultrasound-based cancer immunotherapy.

The present invention also provides a method of preventing or treating cancer, which comprises administering a composition comprising the polymer composite as an active ingredient to a subject.

The present invention also provides a use of a composition comprising the polymer composite as an active ingredient for preventing or treating cancer.

The present invention also provides a use of a composition comprising the polymer composite as an active ingredient for preparing a drug for preventing or treating cancer.

In the present invention, the “cancer immunotherapy” may be the generic term for all systems that eliminate cancer cells by immune cells (killer T cell) by inducing an immune response to a tumor specific antigen or a tumor-associated antigen. For example, a method of inducing immunity against antigens may use genes, proteins, viruses, dendritic cells, or the like.

In the present invention, the “immunogenic cell death (ICD)” means a type of cell death caused by a cell proliferation inhibitor such as anthracyclines, oxaliplatin and bortezomib, or radiotherapy and photodynamic therapy. Unlike common cell death, the ICD of cancer cells may cause an effective antitumor immune response through the activation of dendritic cells and a specific T cell response thereby.

The “cancer” used herein is the generic term for diseases caused by cells having an aggressive characteristic in which cells divide and grow without a normal growth limit, an invasive characteristic in which cells penetrate into surrounding tissues, and a metastatic characteristic in which cells spread to other parts of the body. The cancer may be, for example, one or more selected from the group consisting of colorectal cancer, breast cancer, prostate cancer, melanoma, lung cancer, head and neck cancer, ovarian cancer, bladder cancer, stomach cancer, esophageal cancer, bile duct cancer, pancreatic cancer, liver cancer, cervical cancer, skin cancer, lymphoma, thyroid cancer, bone marrow cancer, endometrial cancer, and brain tumors. According to one experimental example of the present invention, the cancer may be colorectal cancer, breast cancer, or prostate cancer, but the present invention is not limited thereto.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfate (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention.

The present invention also provides a method of preparing a polymer composite for ultrasound-based cancer immunotherapy, which comprises the following steps:

(a) mixing a porphyrin-based sonosensitizer solution and a biocompatible polymer solution to allow a reaction;

(b) adding the reaction solution to a dialysis membrane to perform dialysis;

(c) after dialysis, lyophilizing the amphipathic polymer compound solution in which the biocompatible polymer and the sonosensitizer are combined to prepare a powder; and

(d) encapsulating an peroxalate derivatives in the amphipathic polymer compound using an oil-in-water emulsion method.

In the present invention, (a) may be a step of combining a carboxyl group (—COOH) of the sonosensitizer with an amino group (—NH₂) of the biocompatible polymer by mixing a porphyrin-based sonosensitizer solution and a biocompatible polymer solution.

Here, in the polymer composite, with respect to the biocompatible polymer, the sonosensitizer may be combined in a dry weight ratio of 0.05 to 0.5 times, for example, 0.05 to 0.45 times, 0.05 to 0.4 times, 0.05 to 0.3 times, 0.05 to 0.2 times, 0.1 to 0.5 times, 0.1 to 0.45 times, 0.1 to 0.4 times, 0.1 to 0.3 times, or 0.1 to 0.2 times, but the present invention is not limited thereto.

In addition, in the polymer composite, the biocompatible polymer and the sonosensitizer may be combined in a molar ratio of 1:0.2 to 5, 1:0.2 to 3, 1:0.2 to 2, 1:0.2 to 1.5, 1:0.2 to 1, 1:0.5 to 5, 1:0.5 to 3, 1:0.5 to 2, 1:0.5 to 1.5, 1:0.5 to 1, or 1:1, but the present invention is not limited thereto. According to one example or experimental example of the present invention, the amphipathic polymer compound in which the biocompatible polymer and the sonosensitizer are combined is a material in which the sonosensitizer is conjugated to an end of the linear polymer in a molar ratio of 1:1, and since a size is small, there is an advantage of being relatively easy to penetrate into cancer tissue.

In the present invention, (b) is a step of inputting a reaction solution of the biocompatible polymer and the sonosensitizer to a cellulose dialysis membrane to perform dialysis. Here, the dialysis membrane may have a molecular weight cut off of 1 to 10 kDa, 1 to 8 kDa, 1 to 5 kDa, 1 to 4 kDa, 1 to 3.5 kDa, 2 to 10 kDa, 2 to 8 kDa, 2 to 5 kDa, 2 to 4 kDa, 2 to 3.5 kDa, 3 to 10 kDa, 3 to 8 kDa, 3 to 5 kDa, 3 to 4.5 kDa, 3 to 4 kDa, 5 to 10 kDa, 6 to 8 kDa, or 3.5 kDa, but the present invention is not limited thereto.

In the present invention, (c) is a step of freezing the amphipathic polymer compound solution in which the biocompatible polymer and the sonosensitizer are combined in liquid nitrogen after dialysis and then preparing it in a powder through lyophilization. Here, the amphipathic polymer compound solution may further comprise filtering the solution through a filter having a size of a 0.1 μm to 1.5 μm, 0.1 μm to 1.2 μm, 0.1 μm to 1 μm, 0.3 μm to 1.5 μm, 0.3 μm to 1.2 μm, 0.3 μm to 1 μm, 0.5 μm to 1.5 μm, 0.5 μm to 1.2 μm, 0.5 μm to 1 μm, or 0.8 μm before freezing in liquid nitrogen, but the present invention is not limited thereto.

In the present invention, (d) is a step of dispersing the peroxalate derivatives with a sonicator using an oil-in-water emulsion method and encapsulating it in the amphipathic polymer compound. Here, the peroxalate derivatives may be encapsulated at a molar ratio of 1 to 1000 times, 1 to 900 times, 1 to 800 times, 1 to 700 times, 1 to 600 times, 1 to 500 times, 1 to 400 times, 1 to 300 times, 1 to 200 times, 1 to 150 times, 10 to 1000 times, 10 to 800 times, 10 to 600 times, 10 to 400 times, 10 to 200 times, 10 to 150 times, 50 to 1000 times, 50 to 800 times, 50 to 600 times, 50 to 400 times, 50 to 200 times, 50 to 150 times, 70 to 120 times, or 100 times, with respect to the amphipathic polymer compound in which the biocompatible polymer and the sonosensitizer are combined, but the present invention is not limited thereto.

In one experimental example of the present invention, as a result of evaluating the ROS responsiveness of the polymer composite (Ox@PEG-VPF) of the present invention, it was confirmed that, by the specific reaction between peroxalate and H₂O₂, energy is transferred to VPF and Ce6 to activate, and an aspect in which overall cells swell due to CO₂ generated in cancer cells in which ROS is present at a high concentration was confirmed (see Experimental Example 1). The VPF and Ce6 activated by the energy transfer may increase ROS production.

In another experimental example of the present invention, as a result of confirming the CO₂ generation behavior of the polymer composite according to the present invention, it was confirmed that, when 100 μM H₂O₂ was treated and peroxalate is included, CO₂ is released (see Experimental Example 2).

In still another experimental example of the present invention, as a result of confirming whether CO₂ generation continues depending on the peroxalate and H₂O₂ conditions in the polymer composite according to the present invention using an ultrasound imaging device, it was confirmed that, when peroxalate is included in the polymer composite and 100 μM H₂O₂ is treated, CO₂ generation continues (see Experimental Example 3).

In yet another experimental example of the present invention, it was confirmed that the polymer composite according to the present invention does not exhibit cytotoxicity (see Experimental Example 4).

In yet another experimental example of the present invention, it was confirmed that the peroxalate derivatives in the polymer composite according to the present invention reacts with H₂O₂ to produce CO₂, and CO₂ collapse occurs by cavitation during sonication (see Experimental Example 5).

In yet another experimental example of the present invention, it was confirmed that the amount of ROS production of the sonosensitizer increases during sonication by reaction of the peroxalate derivatives in the polymer composite according to the present invention with H₂O₂ (see Experimental Examples 6 and 7).

In yet another experimental example of the present invention, the effect of killing various types of cancer cells treated with the polymer composite according to the present invention was confirmed, and it was confirmed that the CO₂ cavitation effect and cell membrane rupture are caused by ultrasound, and proteins, which induce immunogenicity, are released outside the cancer cells without degradation, thereby inducing ICD (see Experimental Example 7).

In yet another experimental example of the present invention, when the polymer composite according to the present invention is administered into a tumor-bearing mice, in a group treated with both of a polymer composite comprising an peroxalate derivatives and ultrasound, it was confirmed that the increase in tumor size is significantly inhibited, and the highest survival rate is shown (see Experimental Example 8).

In yet another experimental example of the present invention, when the polymer composite according to the present invention is administered into a cancer-induced mouse, in a group co-treated with a polymer composite comprising an peroxalate derivatives, ultrasound, and an immune checkpoint inhibitor, it was confirmed that the increase in tumor size is most greatly inhibited, and the tumor weight is the smallest, and the expression of antitumor immunity-associated factors is the highest (see Experimental Example 9).

Hereinafter, preferred Examples and Experimental examples for helping the understanding of the present invention will be suggested. However, the following Examples and Experimental examples are provided only to more easily understand the present invention, and the contents of the present invention are not limited by the following Examples and Experimental examples.

### EXAMPLES

### Example 1. Preparation of Polymer Compound Including Sonosensitizer

To formulate a polymer composite that can induce sonodynamic therapy and antitumor immunity, a polymer compound including a sonosensitizer was synthesized.

1-1. Preparation of PEG-VPF Polymer Compound

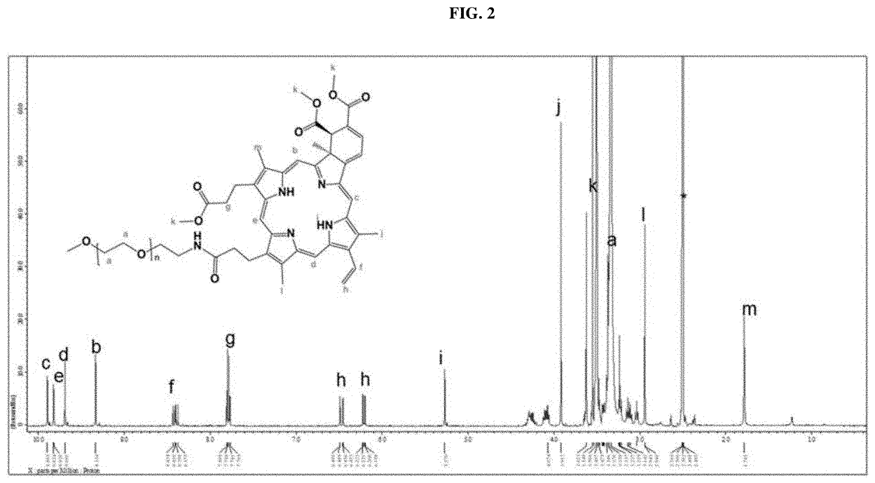

Specifically, 0.03 mmol each of 4-dimethylaminopyridine (DMAP) and 1-ethyl-3-(3-dimethylaminopropyl)carbodiimide (EDC) were dissolved in 1 ml of dimethyl sulfoxide (DMSO), mixed with 0.022 mmol of a verteporfin (VPF) solution dissolved in the same solvent and stirred in a dark place for 3 hours to activate a carboxyl group of VPF. Subsequently, the reaction solution was added dropwise to a 0.02 mmol methoxy PEG amine (mPEG-NH₂, molecular weight: 5 kDa) solution dissolved in a co-solvent in which DMSO and distilled water were mixed in 1:1 ratio to allow a reaction for 24 hours in a light-shielding state. After the termination of the reaction, the reaction solution was put into a cellulose dialysis membrane having a molecular weight cut off of 3.5 kDa to perform dialysis in excess DMSO. The ratio of the dialysis solution changed from 100% DMSO to 100% distilled water for 2 days, and dialysis was further performed in 100% distilled water for 3 days. The purification-completed PEG-VPF solution was frozen in liquid nitrogen through a 0.8-μm filter and then lyophilized to obtain a green powder-type polymer compound (PEG-VPF) containing VPF as a sonosensitizer. The structure of PEG-VPF and its ¹H-NMR analysis result are shown in FIG. 2.

1-2. Preparation of PEG-CMD-Ce6 Polymer Compound

A PEG-CMD-Ce6 conjugate was synthesized in a simple two-step process of conjugating mPEG-NH₂ to carboxymethyl dextran (CMD) and chemically conjugating chlorin e6 (Ce6) to a CMD backbone. mPEG-NH₂ (50 mg, 0.01 mmol) and CMD (150 mg, 0.01 mmol) were dissolved in 0.1 M borate buffer (pH 8.0; 2%, w/v). And then, after adding sodium cyanoborohydride (2.52 mg, 0.04 mmol), the resulting solution was stirred at 45° C. for 4 days. The solution was dialyzed against distilled water using a dialysis membrane for 3 days [molecular weight cut-off=6 to 8 kDa; Spectrum Laboratories Inc., CA, USA], and lyophilized. As the polymer conjugate was dissolved in deuterium oxide (D₂O), the chemical structure of mPEG-NH₂-conjugated CMD (mPEG-CMD) was characterized using a ¹H NMR Varian Unity 500 MHz FT-NMR spectrophotometer. Ce6 was chemically bonded to the CMD backbone through esterification. mPEG-CMD (100 mg, 0.005 mmol) was dissolved in 5 ml of DMSO/distilled water (1:1, v/v), and stirred at 25° C. for 30 minutes. For the activation of a carboxyl group of Ce6, Ce6 (19 mg, 0.032 mmol), EDC HCl (8.05 mg, 0.042 mmol), and DMAP (5.08 mg, 0.042 mmol) were dissolved in 2 ml of DMSO/DMF (1:1, v/v), and stirred for 3 hours in a dark place at 25° C. The two solutions were mixed, and reacted at room temperature for 24 hours under dark conditions. The PEG-CMD-Ce6 solution was dialyzed against a series of methanol and distilled water (1:1, v/v), and sequentially dialyzed against distilled water using a dialysis membrane (molecular weight cut-off=3.5 kDa) for 1 and 2 days. The purified solution was filtered (0.8-mm syringe filter) and lyophilized to obtain a green powder. The chemical structure of the PEG-CMD-Ce6 conjugate was confirmed using ¹H NMR in D₂O/DSMO-d₆ (1:1, v/v).

### Example 2. Preparation of Polymer Composite Including Peroxalate Derivatives

A polymer compound in which a hydrophilic polymer, PEG, and a hydrophobic sensitizer, VPF, were combined in a molar ratio of 1:1 (mole) was amphipathic and is self-assembled in an aqueous environment to form nano-sized particles. For the polymer composite (Ox@PEG-VPF) including dibutyl oxalate according to the present invention, an oil-in-water (o/w) emulsion method was adopted to encapsulate the hydrophobic dibutyl oxalate in the hydrophobic interior of PEG-VPF.

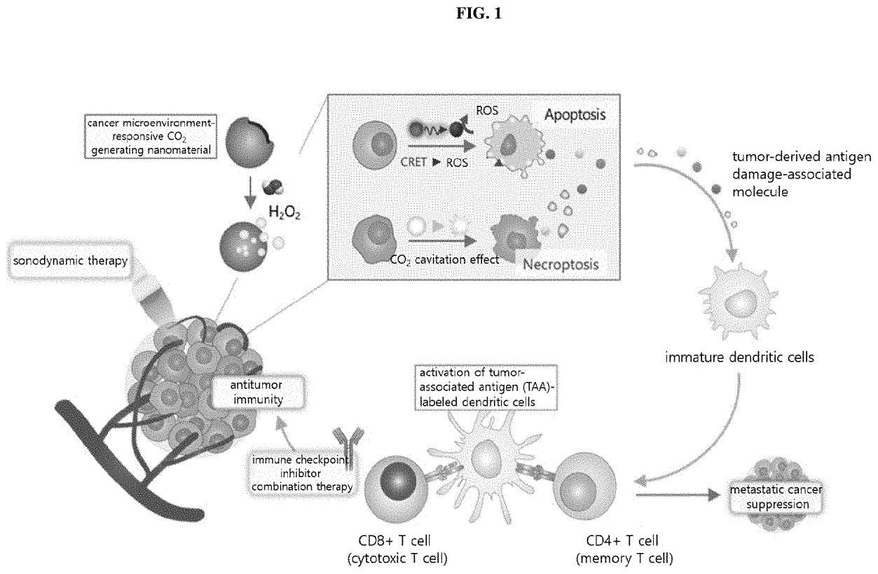

Specifically, 3 mg of the dry PEG-VPF powder prepared in Example 1-1 was dissolved in dichloromethane at a concentration of 40 mg/ml, 10 μl of dibutyl oxalate was added and evenly dispersed for 10 seconds (amplitude: 23%, pulse: 2 s/1 s) using a probe-type sonicator. While sonication continued for 1 minute under the same conditions, 1 ml of distilled water or PBS was slowly added dropwise to emulsify, rapidly stirred in a light-shielded state to remove dichloromethane, thereby preparing the final dosage form of a polymer composite (Ox@PEG-VPF) including dibutyl oxalate. The structure of Ox@PEG-VPF and its functions are illustrated in FIG. 1.

In addition, to encapsulate bis(2,4,6-trichlorophenyl) oxalate (TCPO) in the PEG-CMD-Ce6 conjugate prepared in Example 1-2, each solution was uniformly mixed using an oil-in-water emulsion method. Subsequently, 5 ml of distilled water was added dropwise to the mixed solution, followed by sonication (Sonics Vibra-Cell VCX 750, CT, USA) for 2 minutes (amplitude: 25%, on: 3 sec, off: 1 sec). Then, methylene chloride was evaporated by vigorous stirring under dark conditions, and a polymer composite including TCPO in PEG-CMD-Ce6 was prepared.

### EXPERIMENTAL EXAMPLE

### Experimental Example 1. ROS Responsiveness of Polymer Composite

1-1. Evaluation of ROS Responsiveness of Polymer Composite Using IVIS Imaging System

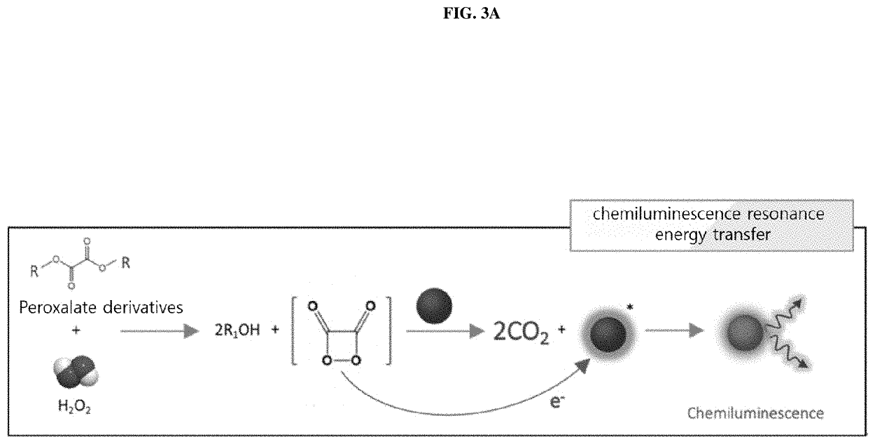

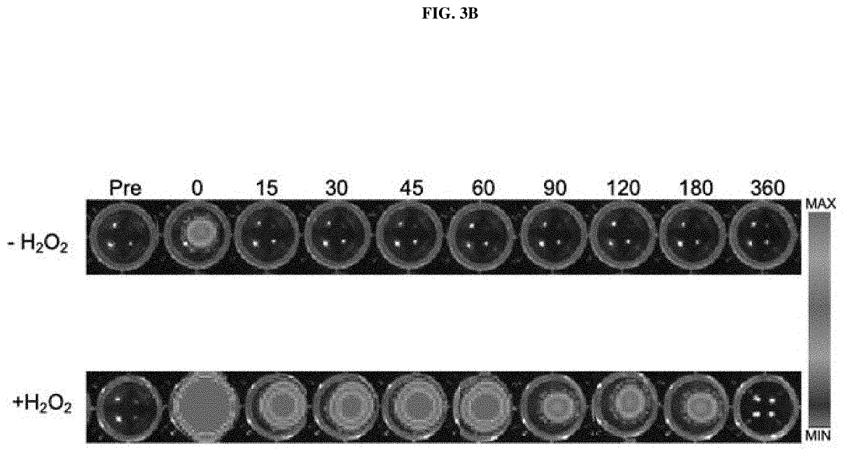

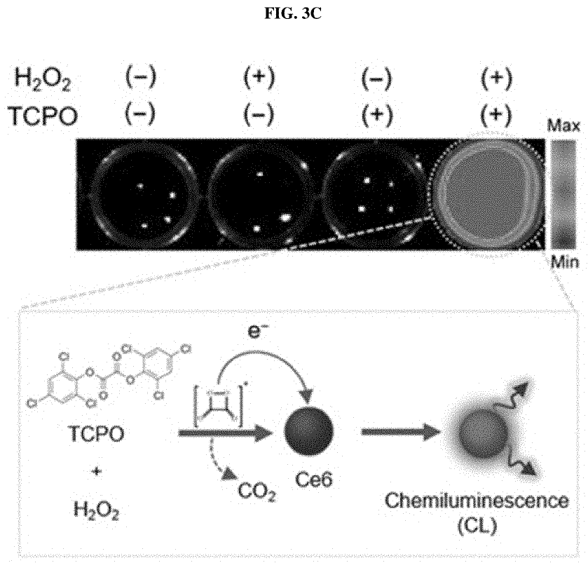

The schematic diagram of chemiluminescence resonance energy transfer by chemical reaction between an peroxalate derivatives and H₂O₂ is shown in FIG. 3A.

The reaction between peroxalate compound and H₂O₂ caused electron transfer as the bond was broken, and in the present invention, a sonosensitizer, VPF, served as a receptor for electrons generated, and it could be observed that chemiluminescence occurs due to chemiluminescence resonance energy transfer.

In addition, as shown in FIGS. 3B and 3C, as a result of observing the chemiluminescence generated depending on 100 μM H₂O₂ treatment, which is a H₂O₂ mimicking condition of tumor tissue using an IVIS imaging system, it was confirmed through the chemiluminescence continuing up to 3 hours in the H₂O₂-containing environment that the polymer composite (Ox@PEG-VPF) according to the present invention (FIG. 3B) and the TCPO-containing polymer composite (FIG. 3C) are activated by energy transfer to VPF and Ce6 by a specific reaction between peroxalate and H₂O₂. Therefore, it was seen that the polymer composite according to the present invention has high ROS responsiveness.

1-2. Evaluation of ROS Responsiveness of Polymer Composite According to In Vitro Cellular Experiment

A colorectal cancer cell line CT26 and a normal cell line L929 were seeded in 60-pi glass bottom cell culture plates and cultured for 24 hours, and then Ox@PEG-VPF corresponding to 5 μM, which is the VPF concentration standard, was diluted in an FBS-free RPMI 1640 culture medium, followed by treatment of the cells. From 15 minutes after the treatment of the polymer composite, changes occurring in the cells were observed in real-time using a microscope.

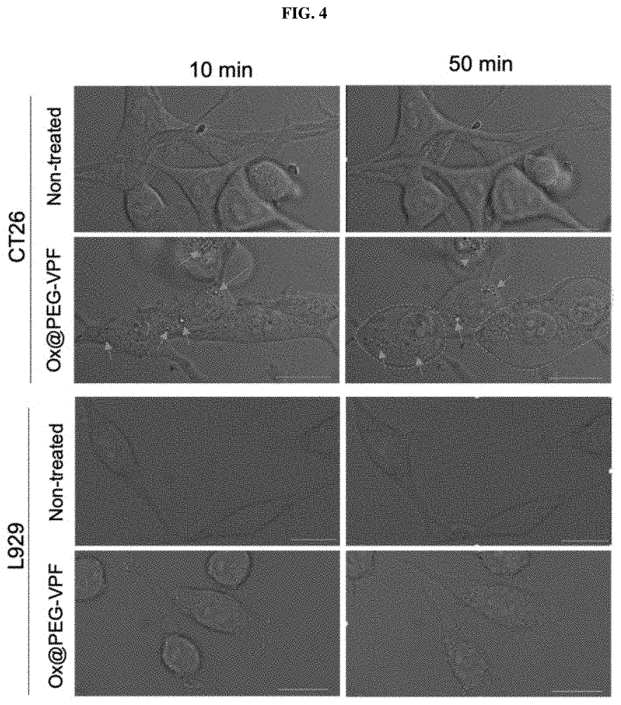

As a result, as shown in FIG. 4, in the case of cancer cells with a high concentration of ROS, as the peroxalate reacted, CO₂ was generated, and a large amount of air bubbles were generated (yellow arrow), confirming that the cells swell as a whole (yellow dotted line).

### Experimental Example 2. Evaluation of CO₂ Generation Behavior Depending on Presence of H₂O₂ and Peroxalate

A polymer composite prepared in the same manner as in Example 2 was 30-fold diluted in a PBS (pH 7.4) solution (0.1 mg/ml PEG-VPF) and sealed after the addition of H₂O₂, and for CO₂ titration, colorimetric analysis of the CO₂ concentration in the solution over time was performed using an activator provided in the kit.

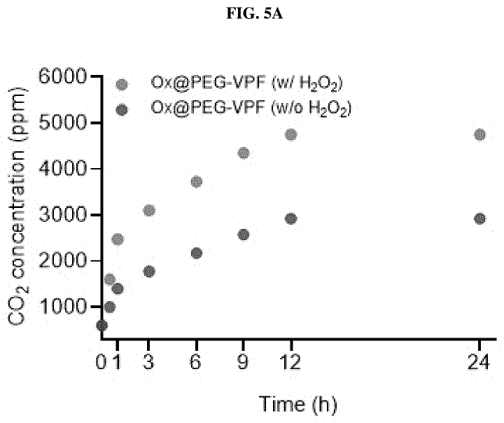

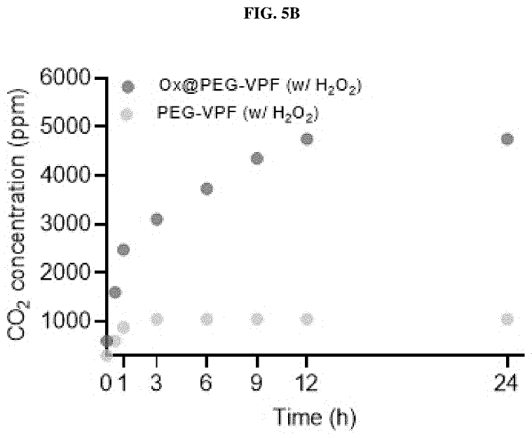

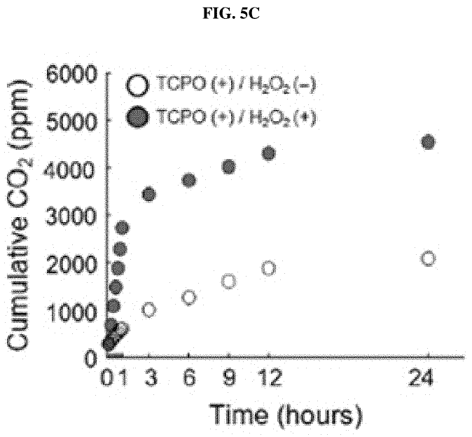

As a result, as shown in FIG. 5A, from the CO₂ generation behavior of the polymer composite Ox@PEG-VPF including dibutyl oxalate according to the present invention, it was confirmed that, when 100 μM H₂O₂ was treated (red dot), 1.77-fold higher CO₂ was generated during the first hour, compared to when not treated (blue dot), and as shown in FIG. 5C, it was confirmed that CO₂ was also generated when a polymer composite including TCPO was treated with H₂O₂.

In addition, as shown in FIG. 5B, when the CO₂ generation behavior by 100 μM H₂O₂ was observed depending on whether the peroxalate was encapsulated in the polymer composite, it was confirmed that CO₂ is released only from the peroxalate-including Ox@PEG-VPF.

### Experimental Example 3. Evaluation of CO₂ Production Using Ultrasound Imaging

Using an ultrasound imaging device (B-mode (US), harmonic mode (Har)), whether CO₂ generation continues depending on the peroxalate and H₂O₂ conditions in the polymer composite was observed.

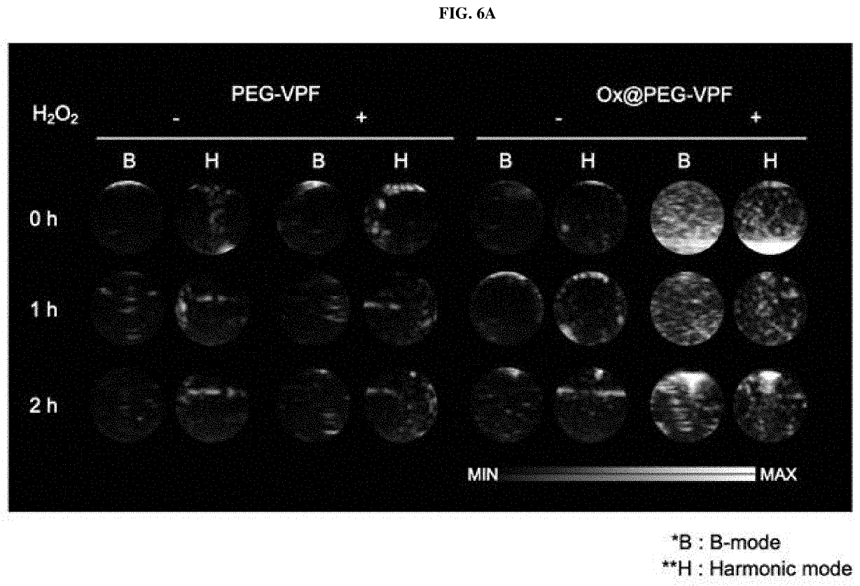

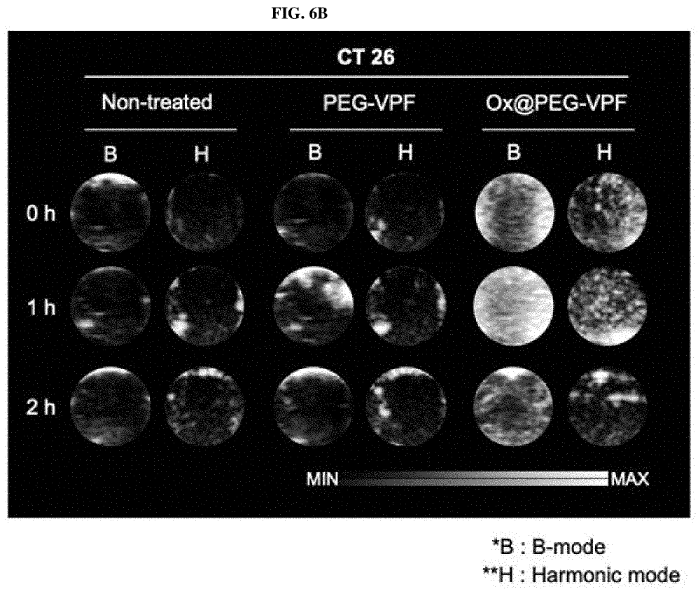

As a result, as shown in FIG. 6A, based on 5 μM VPF in the prepared Ox@PEG-VPF, when 100 μM H₂O₂ was treated in an experimental group in which Ox@PEG-VPF was dispersed in phosphate buffer saline (PBS), it was confirmed that a strong image signal was generated by CO₂ and maintained for approximately 2 hours.

In addition, as a result of observing the suspension of the colorectal cancer cell line CT26 treated with Ox@PEG-VPF and PEG-VPF and cultured for 1 hour, as shown in FIG. 6B, it was confirmed that, when Ox@PEG-VPF was treated, a strong ultrasound signal is generated.

The B-mode (US) and Harmonic mode (Har) shown in each drawing of FIGS. 6A and 6B mean two image measurement methods that can be seen in the device. The B-mode represents all signals sensed by ultrasound, and the Harmonic mode means signals made by bubbles. Referring to FIGS. 6A and 6B, it was seen that CO₂ bubbles specifically responsive to a disease environment were generated in the polymer composite of the present invention.

### Experimental Example 4. Cytotoxicity Evaluation

4-1. In Vitro Evaluation of Cytotoxicity

When the colorectal cancer cell line CT26 and the normal cell line L929 were treated with Ox@PEG-VPF and PEG-VPF for 24 hours at various concentrations based on VPF, the toxicity to cells was evaluated through MTT assay.

The cells were seeded in 96-well plates at a density of 1×10⁴ cells/well, and Ox@PEG-VPF and the control PEG-VPF were treated based on 1 to 10 μM of VPF, and 24 hours later, cell viability was measured compared to cells which had been cultured without treatment.

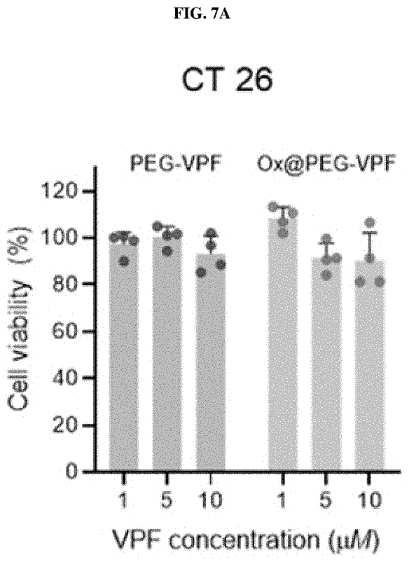

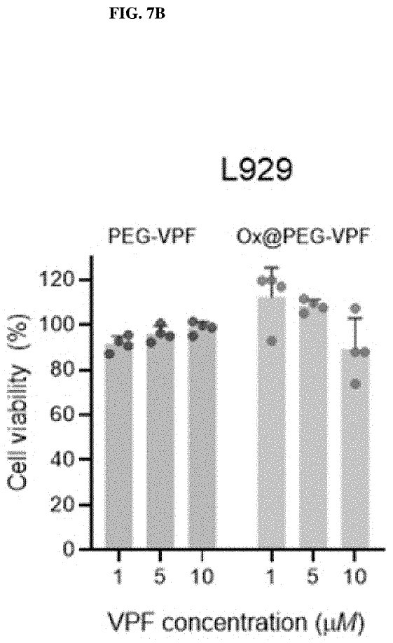

As a result, as shown in FIG. 7A (CT26) and FIG. 7B (L929), it was confirmed that a survival rate of 80% or more was exhibited regardless of peroxalate encapsulation and the type of cell line.

4-2. Cytotoxicity Evaluation According to Ultrasound Irradiation

When a suspension of CT26 which had not been treated in Experimental Example 4-1 was treated with ultrasound under various conditions for 5 minutes, a CCK-8 assay was performed to determine ultrasound conditions that did not affect cells.

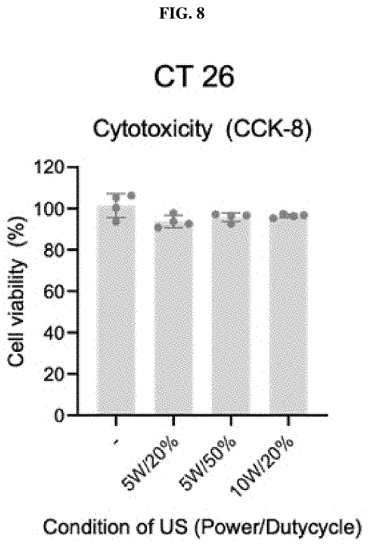

As a result, as shown in FIG. 8, it was confirmed that none of them exhibited cytotoxicity when only ultrasound was treated under a condition of 5 W (power)/20% (duty cycle), 5 W/50%, or 10 W/20%.

### Experimental Example 5. Evaluation of CO₂ Cavitation According to Ultrasound Irradiation

In order to set an appropriate peroxalate ratio in the polymer composite of the present invention, Ox@PEG-VPF and confirm whether CO₂ collapse caused by the cavitation effect occurs, various molar rations of the composite were prepared, followed by observing non-linear ultrasound images.

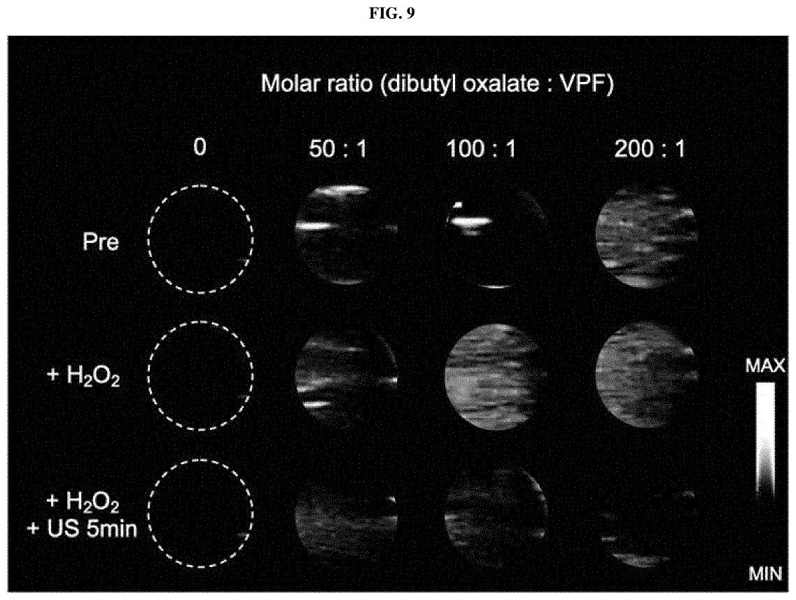

As a result, as shown in FIG. 9, when Ox@PEG-VPF in which excess peroxalate, which is 100-fold (molar ratio) larger than PEG-VPF, was encapsulated, in a 100 μM H₂O₂ environment, which is a cancer tissue mimicking condition, selective CO₂ was produced, and by the ultrasound treatment based on FIG. 8 (power 5 W, duty cycle 50%, pulse repetition frequency (PRF) 1 Hz, 5 min), the dissipation of image signals caused by the rupture of CO₂ bubbles was observed.

From the above, as a result of having no image signal (indicating that no CO₂ is generated) is generated before H₂O₂ treatment, losing the image signal because of cavitation and bubble collapse after ultrasound treatment, and satisfying the condition of as much containing peroxalate as possible, the molar ratio of the peroxalate was finally selected to be 100 times that of PEG-VPF.

### Experimental Example 6. Evaluation of Ability to Generate ROS

To confirm whether ROS is generated by the polymer composite, Ox@PEG-VPF, by treating the ultrasound, according to the present invention, N,N-dimethyl-4-nitrosoaniline (RNO) was used. When the peroxalate and H₂O₂ were reacted in Ox@PEG-VPF, because the ROS production amount of the sonosensitizer may increase, all experiments were performed under a cancer microenvironment mimicking condition, which is the 100 μM H₂O₂ condition.

For RNO assay, 20 μl of the Ox@PEG-VPF prepared in Example 2 was prepared by diluting 1.980 ml of a mixed solution of 100 μM H₂O₂ and 10 μM RNO. After 2 ml of the mixed solution was put into an agarose mold and treated by ultrasound treatment for 5 minutes, the ability to produce ROS was evaluated using the fact that the absorbance of RNO is reduced when VPF is exposed to ultrasound to generate ROS, and the absorbance of RNO decreased in the wavelength band of 440 nm was measured using an UV/Vis spectrophotometer. Here, the standard of initial absorbance was the mixed solution of 100 μM H₂O₂ and 10 μM RNO.

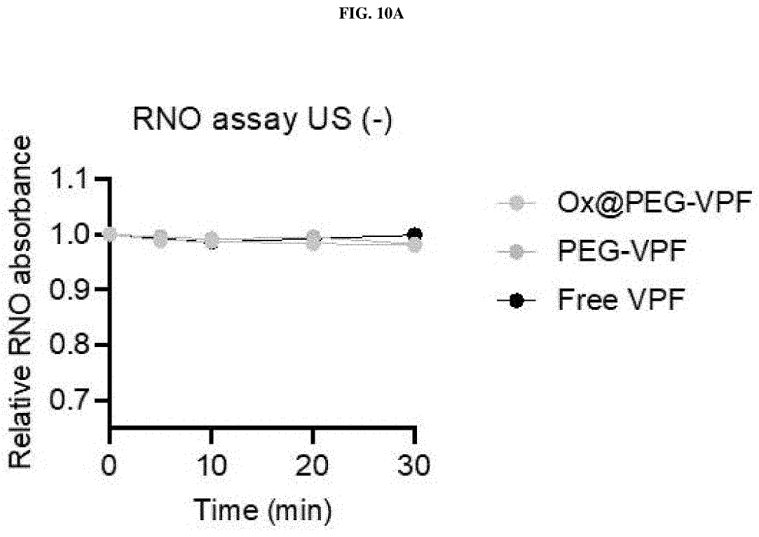

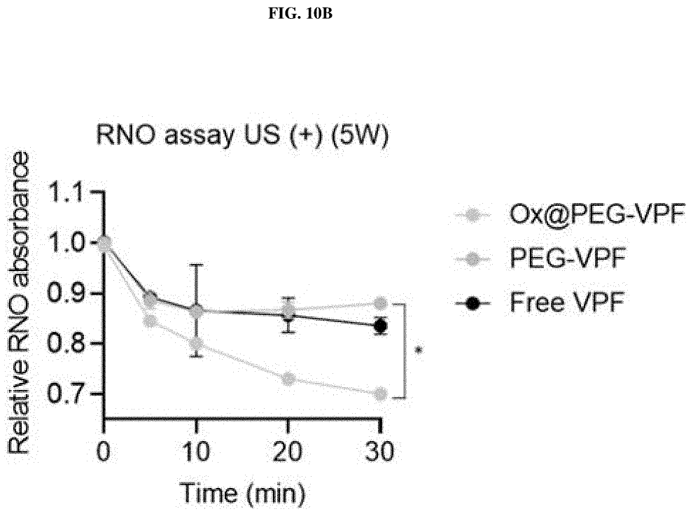

As a result, as shown in FIG. 10A, when ultrasound treatment was not performed, ROS was not generated in both Ox@PEG-VPF and the control, and as shown in FIG. 10B, it was confirmed that, in the case of Ox@PEG-VPF treated with ultrasound, 2.06- and 2.58-fold more ROS were produced with respect to the controls containing the same concentration of the VPF, such as VPF and PEG-VPF, respectively.

### Experimental Example 7. Confirmation of Ability to Generate ROS and ICD In Vitro

CT26 colorectal cells were cultured in an RPMI 1640 medium containing 10% fetal bovine serum (FBS) and 1% antibiotic-antimycotic. 5×10⁶ of CT26 cells were treated with Ox@PEG-VPF and the controls for 2 hours based on 5 μM VPF, and then resuspended in 1 ml of the RPMI 1640 medium, followed by ultrasound treatment for 5 minutes.

7-1. Evaluation of Ability to Generate ROS Using DCF-DA

After 100 μl of the CT26 cell suspension that had undergone ultrasound treatment was mixed in 900 μl of a 10 μM DCF-DA-added RPMI 1640 medium and treated for 30 minutes, the resulting mixture was washed twice using PBS and observed by confocal laser scanning microscopy.

Specifically, the colorectal cancer cell line, CT26, was treated with experimental groups containing VPF and peroxalate based on 5 μM VPF and cultured for 2 hours, and then whether ROS is generated according to ultrasound application was measured using 2′,7′-dichlorofluorescein diacetate (DCF-DA) and confocal microscopy.

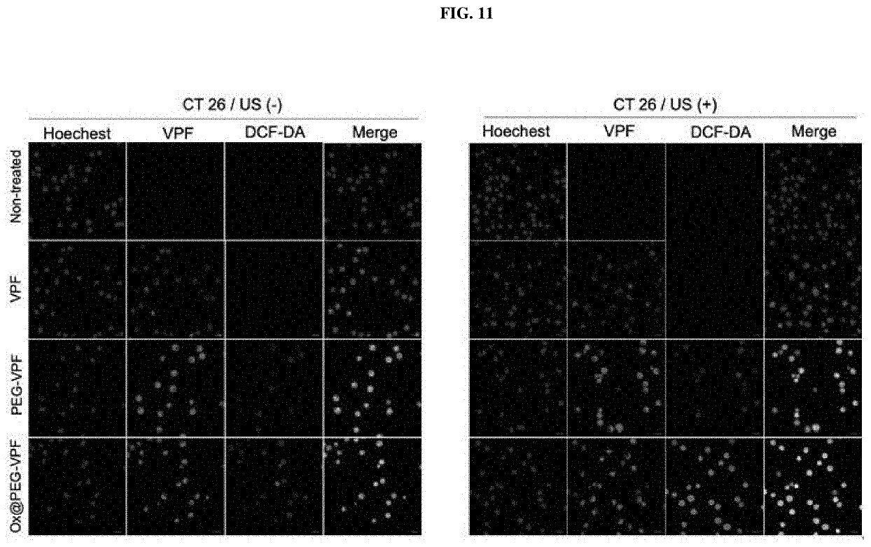

As a result of evaluating the ability to generate ROS in cancer cells of Ox@PEG-VPF using confocal microscopy, as shown in FIG. 11, DCF-DA exposed to ROS was oxidized to qualitatively/quantitatively analyze the amount of ROS generation by the sonodynamic effect using a molecular probe with a green fluorescence signal, and it was confirmed that the experimental group treated with both of ultrasound and Ox@PEG-VPF exhibits a strong fluorescence signal compared to peroxalate PEG-VPF in which peroxalate was not encapsulated.

This means that, despite containing the same amount of a sonosensitizer, more ROS was produced by increasing the activity of VPF by the chemical reaction between H₂O₂ and peroxalate.

7-2. Evaluation of Cytotoxicity According to Ultrasound Application

To induce SDT and ICD, the cytotoxicity that occurs when irradiated with ultrasound from the outside was evaluated.

To this end, a colorectal cancer cell line CT26, a breast cancer cell line 4T1, and a prostate cancer cell line PC3 were treated with Ox@PEG-VPF based on 5 μM VPF, and a cell suspension that had been incubated for 2 hours was treated with ultrasound for 5 minutes. Afterward, after the cells were seeded in a 96-well plate and cultured for 6 hours, cytotoxicity was evaluated by CCK-8 assay.

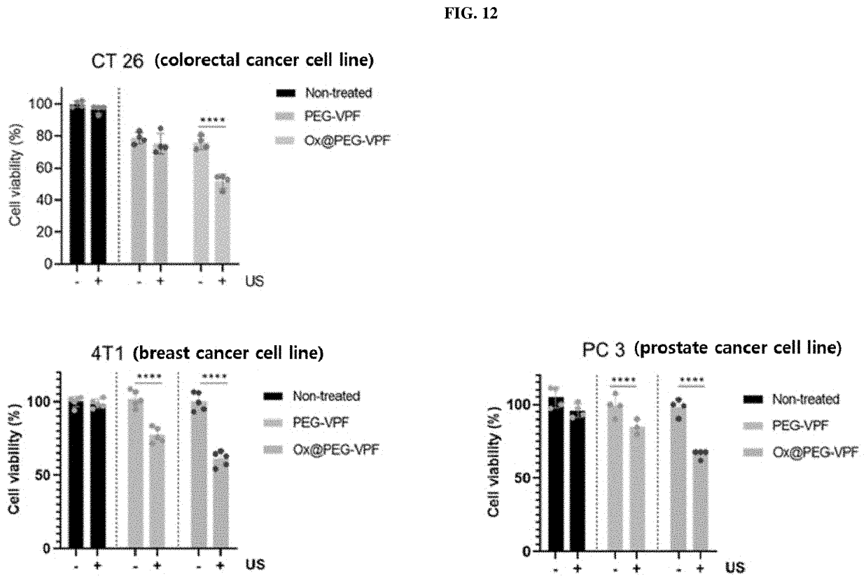

As a result, as shown in FIG. 12, it was confirmed that, when each Ox@PEG-VPF-treated cancer cell was irradiated with ultrasound, compared to untreated cells, 30 to 40% or more cancer cells were killed, and the cell death rate was more than twice as high as that of PEG-VPF treated with the same amount of VPF.

7-3. Evaluation of Cell Death Type by Annexin V and PI Staining

After 100 μl of the CT26 cell suspension that had been treated with ultrasound was resuspended in 900 μl of the FITC-annexin V/PI mixed solution and maintained for 30 minutes, the resulting cells were washed twice with PBS and observed by confocal microscopy.

Specifically, the Ox@ PEG-VPF of the present invention reacted with a high concentration of ROS present in cancer cells to produce CO₂ in the cells, and was treated with a FITC-labeled annexin compound (FITC-annexin V; excitation/emission wavelength—490/525 nm) and propidium iodide (PI; excitation/emission wavelength 535/617 nm) to confirm whether ICD occurs by the cavitation effect and cell membrane rupture caused by ultrasound.

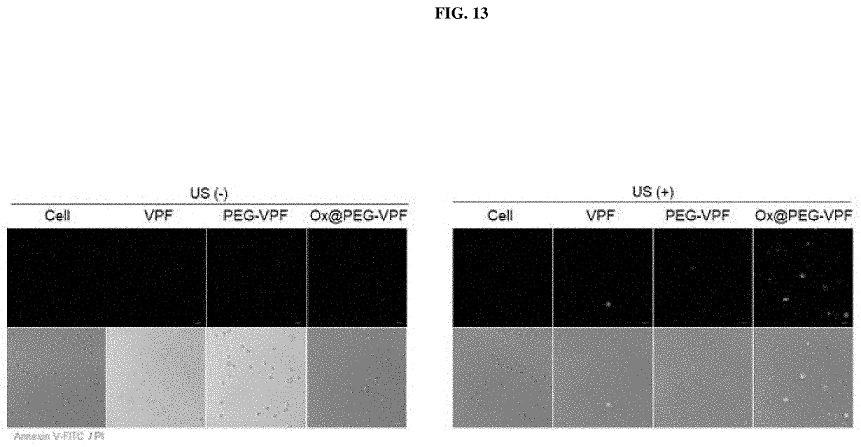

As a result of evaluating ICD caused by ultrasound and the CO₂ cavitation effect, as shown in FIG. 13, when both of ultrasound and VPF were treated, ROS generated from VPF induced the apoptosis of cancer cells, and a FITC-annexin V stain (green fluorescence) indicating cell membrane debris because of cell membrane damage was able to be confirmed, and it was confirmed that PI penetrating into the cells exhibited fluorescence.

Particularly, in the case of cancer cells treated with Ox@PEG-VPF and ultrasound, it was confirmed that the disrupted cell fragments were distributed throughout the image while being bound with FITC-annexin V, indicating that the physical rupture of the cell membrane occurred by the CO₂ bursting by ultrasound. That is, the experimental result showed the possibility of Ox@PEG-VPF to induce cancer cell-selective ICD.

7-4. Evaluation of Cell Death Type Using Western Blotting

For ICD, unlike apoptosis, related proteins that can activate the body's immune response in cancer cells must be leaked out of the cells without digestion. Therefore, western blotting was used to confirm whether the Ox@PEG-VPF of the present invention can cause not only apoptosis caused by a sonodynamic effect, but ICD caused by CO₂.

To this end, 100 μl of a CT26 cell suspension that had been irradiated with ultrasound was resuspended in 4 ml of an FBS-free RPMI 1640 medium, and then incubated in a 100-pi culture dish for 6 hours. Afterward, the supernatant was collected, centrifuged at 1500 rpm for 3 minutes and then cell debris was removed, followed by concentration using a centrifugal filter.

The cells obtained by culture medium removal were completely lysed using a buffer solution for radioimmunoprecipitation assay (RIPA), and centrifuged at 14,000 rpm for 20 minutes, thereby obtaining a supernatant except the remaining debris as a sample. Proteins in each sample were quantified by a bicinchoninic acid (BCA) method, and a sample having the same protein concentration was prepared to detect apoptosis- and ICD-related proteins.

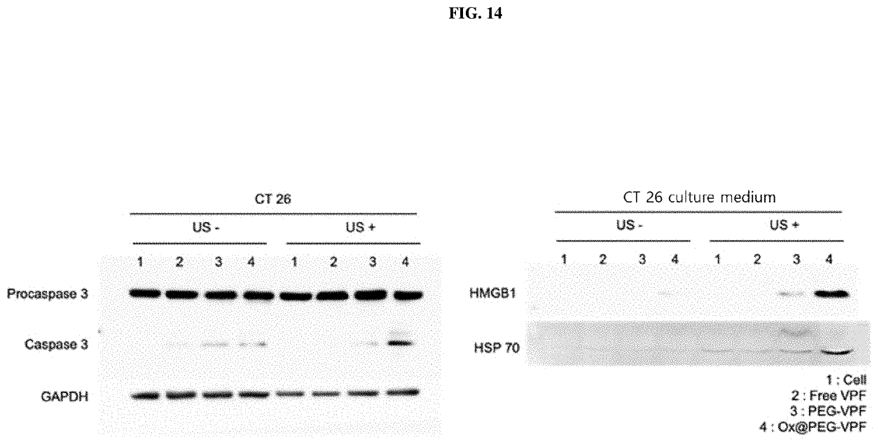

As a result, as shown in FIG. 14, in the Ox@PEG-VPF-treated group, a cleavage caspase-3 band associated with the apoptosis caused by the sonodynamic effect was able to be confirmed. In addition, from the cell culture-concentrated sample, the bands of high mobility group box 1 (HMGB1), which is a type of DAMP, and heat shot protein 70 (HSP70), which were not oxidized by a protease, were able to be confirmed. That is, it was confirmed that not only apoptosis was induced by Ox@PEG-VPF and ultrasound treated in cancer cells, but also ICD was induced by the release of related proteins to activate an immune response in the body outside the cells.

### Experimental Example 8. Evaluation of Anticancer Effect by Ultrasound Treatment In Vivo

To confirm the anticancer effect of Ox@PEG-VPF according to ultrasound treatment in vivo, the experiment was carried out in the following way.

After a colorectal cancer cell line CT26 was cultured in an RPMI 1640 medium supplemented with 10% FBS and 1% antibiotic-antimycotic, 10⁶ cells were dispersed in 100 μl of a culture medium and then subcutaneously injected into the left thigh of a 5-week-old balb/c mouse, thereby preparing an animal model. After the treatment, a tumor size was checked once every 2 days, and an experiment was conducted when its volume was 80 to 100 mm³.

After the Ox@PEG-VPF according to the present invention was intravenously injected into the mouse based on 2 mg/kg VPF, 6 hours later, 8 treatment points were selected for 60 seconds under an ultrasound condition of 5 W/50%/1 Hz (power/duty cycle/pulse repetition frequency) to treat ultrasound, followed by repeating the above procedure 4 times at every 3 days.

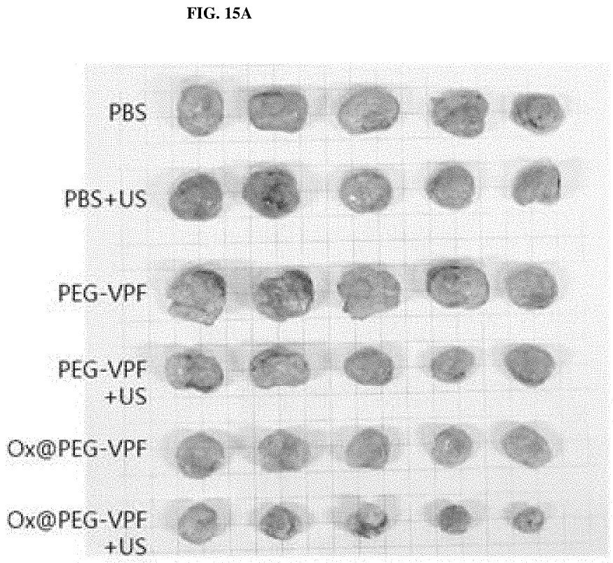

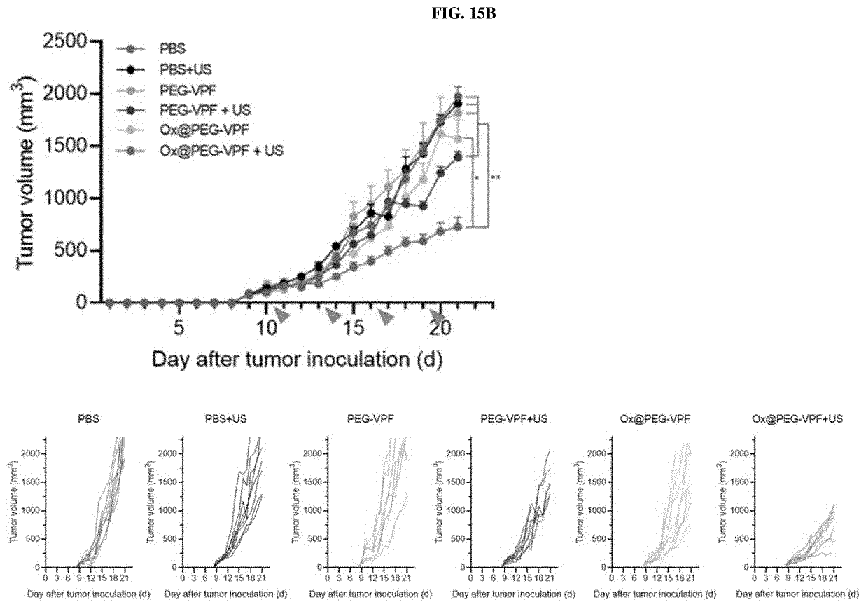

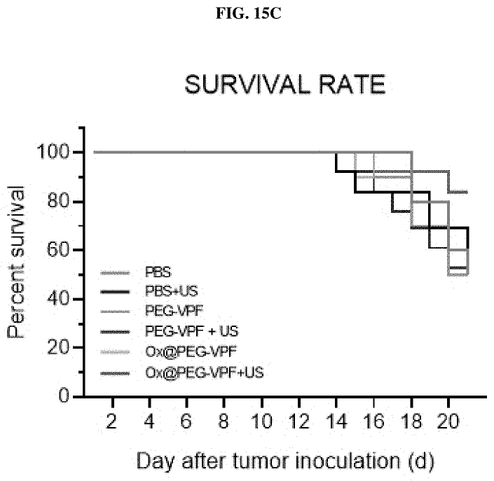

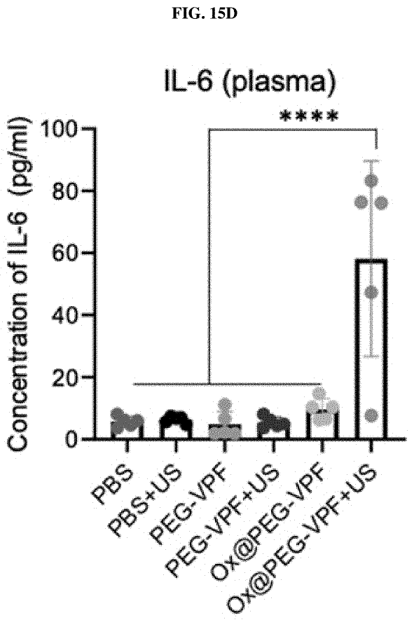

As a result of checking the tumor size of the mouse, as shown in FIG. 15A, the group administered Ox@PEG-VPF and irradiated with ultrasound showed the smallest tumor size compared to other treatment groups, and as a result of confirming the tumor size over time, as shown in FIG. 15B, it was confirmed that the tumor size was increased the least in the Ox@PEG-VPF and ultrasound-treated group.

In addition, as a result of confirming the survival rate of cancer-induce mice, as shown in FIG. 15C, it was confirmed that the group treated with both Ox@PEG-VPF and ultrasound showed the highest survival rate.

In addition, after the treatment of the cancer-induced mice was completed, as shown in FIG. 15D, an immune-associated factor represented by blood interlukin-6 (IL-6) was confirmed by enzyme-linked immunosorbent assay (ELISA), confirming that it is detected at the highest level in the Ox@PEG-VPF and ultrasound-treated group.

### Experimental Example 9. Evaluation of Anticancer Effect and Antitumor Immunity According to Ultrasound Treatment In Vivo

An experiment was conducted when a tumor volume was 80 to 100 mm³ in an animal model prepared in the same manner as in Experimental Example 8.

The Ox@PEG-VPF according to the present invention was intravenously injected into the mouse based on 2 mg/kg VPF, and 6 hours later, 8 treatment points were selected for 60 seconds under an ultrasound condition of 5 W/50%/1 Hz (power/duty cycle/pulse repetition frequency) to treat ultrasound. 24 hours after the ultrasound treatment, 200 μg of a programmed cell death protein 1 (PD-1)-blocking antibody (anti PD-1 antibody) was injected intraperitoneally. The above procedure was repeated 4 times.

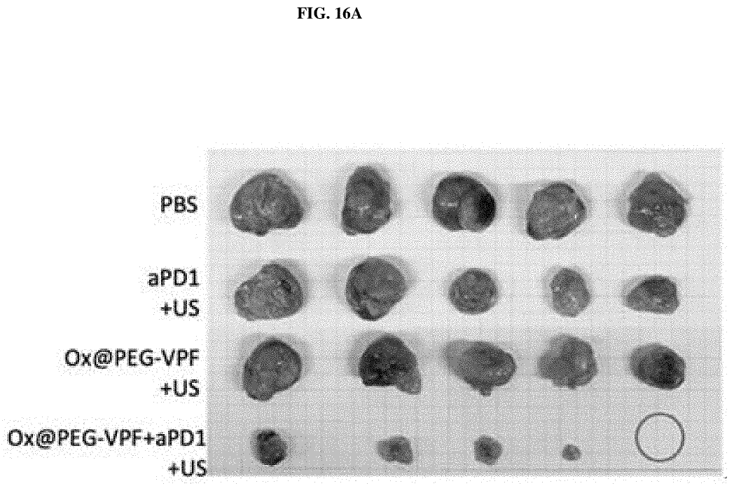

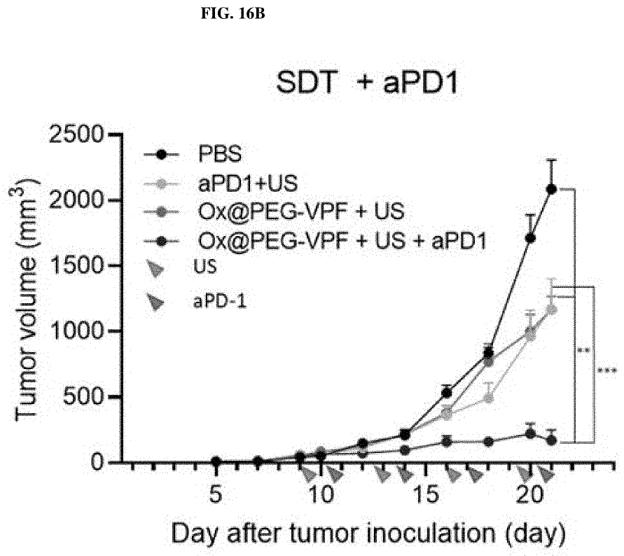

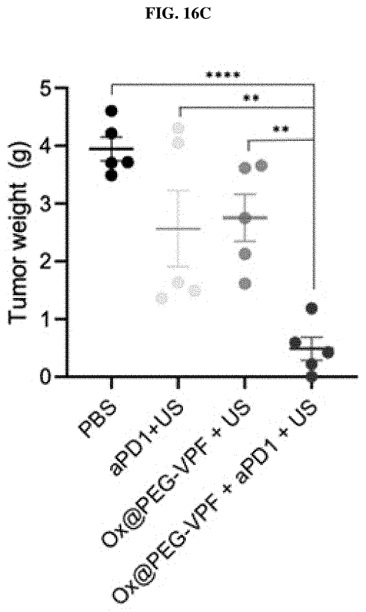

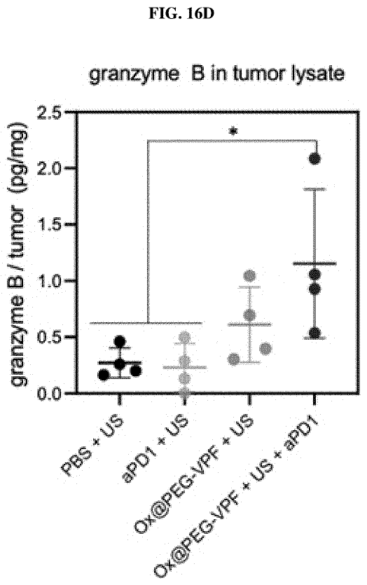

As a result of checking the tumor size of a mouse, as shown in FIG. 16A, when Ox@PEG-VPF was administered and an anti PD-1 antibody as an immune checkpoint inhibitor and ultrasound were treated, compared to other treatment groups, the tumor size was the smallest, and as a result of checking the tumor size over time, as shown in FIG. 16B, in the group treated with Ox@PEG-VPF, ultrasound and an immune checkpoint inhibitor, the tumor size was increased the least.

In addition, as shown in FIG. 16C, the tumor size of the mouse after the treatment was completed was the smallest in the group treated with all of the Ox@PEG-VPF, ultrasound, and the immune checkpoint inhibitor, and as a result of confirming granzyme B, which is an antitumor immunity-associated factor present in tumor tissue, through ELISA, as shown in FIG. 16D, an experimental group treated with all of Ox@PEG-VPF, ultrasound, and an immune checkpoint inhibitor was present at a significantly higher level.

A polymer composite according to the present invention is characterized by peroxalate derivatives being encapsulated in an amphipathic polymer compound in which a biocompatible polymer and a sonosensitizer are combined, wherein the peroxalate derivatives produce free electrons and CO₂ by reaction with a high concentration of H₂O₂ in cancer tissue, the generated electrons raise the energy level of the sonosensitizer in the polymer composite to increase the amount of ROS production, thereby exhibiting an effect of increasing the death rate of cancer cells. In addition, by ultrasound treatment, ICD is induced due to the cavitation effect of the produced CO₂, so molecules capable of activating immune cells in cancer cells are released without damage to induce an immune response to cancer. Therefore, the polymer composite according to the present invention is expected to be effectively used as an ultrasound-based cancer immunotherapeutic agent.

The above-described description of the present invention is provided for illustrative purposes, and those of ordinary skill in the art to which the present invention pertains will understand that the present invention can be easily modified into other specific forms without changing the technical spirit or essential features of the present invention. Therefore, it should be understood that the above-described Examples are illustrative only in all aspects and are not restrictive.

1. A polymer composite for ultrasound-based cancer immunotherapy,
comprising peroxalate derivatives,
wherein the polymer composite is characterized by the peroxalate derivatives being encapsulated in an amphipathic polymer compound in which a biocompatible polymer and a porphyrin-based sonosensitizer are combined.

2. The composite of claim 1, wherein the peroxalate derivatives is one or more selected from the group consisting of dibutyl oxalate, bis(2,4,6-trichlorophenyl) oxalate (TCPO), and bis[2,4,5-trichloro-6-(pentyloxycarbonyl)phenyl] oxalate (CPPD).

3. The composite of claim 1, wherein the biocompatible polymer is one or more selected from the group consisting of polyethylene glycol (PEG), carboxymethyl dextran (CMD), a dextran derivative, and hyaluronic acid.

4. The composite of claim 3, wherein the molecule weight of the PEG is 1 to 500 kDa.

5. The composite of claim 1, wherein the porphyrin-based sonosensitizer is one or more selected from the group consisting of verteporfin (VPF) and chlorin e6.

6. The composite of claim 1, wherein in the polymer composite, the dry weight ratio of the sonosensitizer is 0.05- to 0.5-fold that of the biocompatible polymer.

7. The composite of claim 1, wherein in the polymer composite, the molar ratio of the sonosensitizer is 0.2- to 5-fold that of the biocompatible polymer.

8. The composite of claim 1, wherein the peroxalate derivative is encapsulated at a 1- to 1000-fold molar ratio, with respect to the amphipathic polymer compound in which the biocompatible polymer and the sonosensitizer are combined.

9. The composite of claim 1, wherein the peroxalate derivatives react with hydrogen peroxide (H₂O₂) in cancer tissue to increase reactive oxygen species (ROS) production of the sonosensitizer during ultrasound treatment.

10. The composite of claim 1, wherein the peroxalate derivatives react with H₂O₂ in cancer tissue to produce carbon dioxide (CO₂).

11. The composite of claim 10, wherein the CO₂ causes cavitation during ultrasound treatment, thereby inducing immunogenic cell death (ICD).

12. The composite of claim 1, wherein the polymer composite is released out of cells while one or more immunogenicity-associated proteins selected from the group consisting of tumor-associated antigens (TAAs) and damage-associated molecular patterns (DAMPs) in cancer cells are not degraded.

13. An ultrasound-based cancer immunotherapy method, comprising:
administering a composition comprising the polymer composite of claim 1 as an active ingredient to a subject.

14. The method of claim 13, wherein the composition further comprises an immune checkpoint inhibitor.

15. The method of claim 14, wherein the immune checkpoint inhibitor is one or more selected from the group consisting of a PD-1 inhibitor, a PD-L1 inhibitor, and a CTLA-4 inhibitor.

16. A method of preventing or treating cancer, comprising:
administering a composition comprising the polymer composite of claim 1 as an active ingredient to a subject.

17. The method of claim 16, wherein the cancer is one or more selected from the group consisting of colorectal cancer, breast cancer, prostate cancer, melanoma, lung cancer, head and neck cancer, ovarian cancer, bladder cancer, stomach cancer, esophageal cancer, bile duct cancer, pancreatic cancer, liver cancer, cervical cancer, skin cancer, lymphoma, thyroid cancer, bone marrow cancer, endometrial cancer, and brain tumors.

18. A method of preparing a polymer composite for ultrasound-based cancer immunotherapy, comprising the following steps:
(a) mixing a porphyrin-based sonosensitizer solution and a biocompatible polymer solution to allow a reaction;
(b) adding the reaction solution to a dialysis membrane to perform dialysis;
(c) after dialysis, lyophilizing the amphipathic polymer compound solution in which the biocompatible polymer and the sonosensitizer are combined to prepare a powder; and
(d) encapsulating peroxalate derivatives in the amphipathic polymer compound using an oil-in-water emulsion method.

19. The method of claim 18, wherein, in (b), the dialysis membrane has a molecular weight cut-off of 1 to 10 kDa.
